(19) United States
(12) Patent Application Publication
(10) Pub. No.: US 2026/0056118 A1
(43) Pub. Date: Feb. 26, 2026

(54) IMMUNOASSAY SYSTEM

(71) Applicant: Canon Medical Systems Corporation, Otawara-shi (JP)

(72) Inventors: Sayo KATSUKI, Utsunomiya (JP); Satoshi YAMANE, Nasushiobara (JP); Ichiro TONO, Otawara (JP)

(73) Assignee: Canon Medical Systems Corporation, Otawara-shi (JP)

(21) Appl. No.: 19/302,179

(22) Filed: Aug. 18, 2025

(30) Foreign Application Priority Data
Aug. 21, 2024 (JP) 2024-139787

(51) Int. Cl. G01N 21/27; G01N 33/543; G01N 33/68

(52) U.S. Cl. CPC G01N 21/272; G01N 21/274; G01N 33/543; G01N 33/6854; G01N 2201/122; G01N 2201/126; G01N 2201/12753

(57) ABSTRACT

According to one embodiment, an immunoassay system includes a measurement unit, a calculation unit, and a selection unit. The measurement unit measures a measurement target substance contained in a specimen in accordance with a measurement sequence, and acquires a measurement signal reflecting a concentration of the measurement target substance. The calculation unit calculates an index value related to a fluctuation in intensity of the measurement signal during a first period. The selection unit selects a single measurement sequence to be used in processing during or after the first period in accordance with a concentration range corresponding to an index value of the measurement target substance.

### CROSS-REFERENCE TO RELATED APPLICATION(S)

This application is based upon and claims the benefit of priority from Japanese Patent Application No. 2024-139787, filed Aug. 21, 2024, the entire contents of which are incorporated herein by reference.

### FIELD

Embodiments described herein relate generally to an immunoassay system.

### BACKGROUND

Various techniques are known as a method for measuring a target substance in a biological sample, and various detection methods that employ an antigen-antibody reaction exist. For such detection methods, various techniques such as immunonephelometry as represented by enzyme-linked immune-sorbent assay (ELISA) and latex agglutination, immunochromatography, surface plasmon resonance immunoassay, optical-waveguide immunodetection, etc. are used. In the optical-waveguide immunodetection technique, for example, by detecting a complex formed on a surface of an optical waveguide through attenuation of light, a measurement target substance is measured using the optical waveguide and microparticles on which an antibody, etc. that specifically binds to the measurement target substance is immobilized.

If a high sensitivity is attempted using such a detection technique, quantitative properties may be lost at a high-concentration range, resulting in a narrower dynamic range. Moreover, if an antigen is contained in an excessive amount in a specimen, an inaccurate measurement result may be obtained by the prozone phenomenon by which an apparent measurement value becomes lower. In this manner, it is difficult to achieve both high-sensitivity measurement and wide-range quantitative measurement with respect to test items for which a wide dynamic range related to the concentration is required.

### BRIEF DESCRIPTION OF THE DRAWINGS

FIG. 1 is a diagram showing a configuration example of an immunoassay system according to a first embodiment.

FIG. 2 is a diagram showing a principle of optical measurement of an optical measurement device.

FIG. 3 is a diagram showing temporal changes of an intensity of a measurement signal by comparison between a case of measurement of a high-concentration antigen and a case of measurement of a low-concentration antigen.

FIG. 4 is a diagram showing a procedure for optical measurement with the immunoassay system according to the first embodiment.

FIG. 5 is a diagram showing an example of a display screen for quantitative values according to the first embodiment.

FIG. 6 is a diagram showing a configuration example of an immunoassay system according to a second embodiment.

FIG. 7 is a diagram showing a procedure for performing optical measurement with the immunoassay system according to the second embodiment.

FIG. 8 is a diagram showing an example of a display screen for quantitative values according to the second embodiment.

FIG. 9 is a diagram showing a procedure for optical measurement with an immunoassay system according to a third embodiment.

FIG. 10 is a diagram showing a procedure for optical measurement with an immunoassay system according to a third embodiment.

FIG. 11 is a diagram showing an example of a display screen for quantitative values according to the third embodiment.

FIG. 12 is a diagram showing a measurable concentration range for each measurement condition according to the first embodiment.

FIG. 13 is a diagram showing a measurable concentration range for each measurement condition according to the second embodiment.

FIG. 14 is a diagram showing a measurable concentration range for each measurement condition according to the third embodiment.

### DETAILED DESCRIPTION

An immunoassay system according to an embodiment includes a measurement unit, a calculation unit, and a selection unit. The measurement unit measures a measurement target substance contained in a specimen in accordance with a measurement sequence, and acquires a measurement signal reflecting a concentration of the measurement target substance. The processing circuitry calculates an index value related to a fluctuation in intensity of the measurement signal during a first period. The selection unit selects a single measurement sequence to be used in processing during or after the first period in accordance with a concentration range corresponding to the index value of the measurement target substance.

Hereinafter, an immunoassay system according to the present embodiment will be described in detail with reference to the accompanying drawings. The immunoassay system according to the present embodiment is applicable to any system capable of optically measuring the measurement target substance by employing the antigen-antibody reaction, such as immunonephelometry as represented by enzyme-linked immune-sorbent assay (ELISA) and latex agglutination, immunochromatography, surface plasmon resonance immunoassay, optical-waveguide immunodetection, etc. The principle of optical measurement is not particularly limited to a specific one. Also, in an aspect using magnetic particles, measurement of the measurement target substance is not limited to optical measurement, and magnetic or electromagnetic wave measurement may be adopted. It is assumed that some of the embodiments to be described below adopt, as an example of a principle for quantifying the measurement target substance, an optical-waveguide immunodetection method in which optical measurement is performed using magnetic particles to which an antibody that specifically binds to the measurement target substance is bound.

### First Embodiment

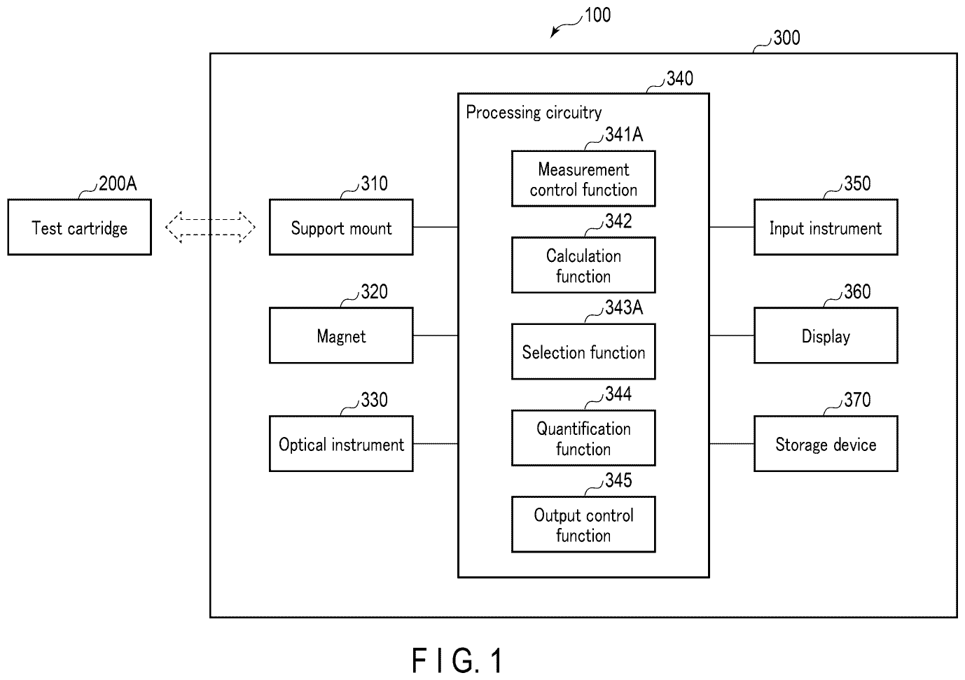

FIG. 1 is a diagram showing a configuration example of an immunoassay system 100 according to a first embodiment. The immunoassay system 100 is a system for optically measuring a measurement target substance by employing an optical-waveguide immunodetection method that uses magnetic particles. The measurement target substance is, for example, an antigen such as an influenza virus, an adenovirus, a respiratory syncytial (RS) virus, a coronavirus (e.g., COVID-19), etc., but is not limited thereto, and may be any substance that can be detected by the immunoassay system 100. As shown in FIG. 1, the immunoassay system 100 includes a test cartridge 200A and an optical measurement device 300.

The test cartridge 200A includes a substrate (hereinafter referred to as a “translucent substrate”) on which a first substance that specifically binds to the measurement target substance is immobilized. The test cartridge 200A includes a drop hole that communicates with a reaction tank provided therein. A mixed liquid of magnetic particles and a specimen treatment liquid in which a specimen (a biological sample) containing the measurement target substance is suspended is dropped into the reaction tank via the drop hole. As the specimen treatment liquid, a buffer solution containing a surfactant agent, for example, is used. A second substance that specifically binds to the measurement target substance is bound to the magnetic particles. A mixed liquid of the specimen treatment liquid, the measurement target substance, and the magnetic particles will be referred to as a “test solution”.

As shown in FIG. 1, the optical measurement device 300 includes a support mount 310, a magnet 320, an optical instrument 330, processing circuitry 340, an input instrument 350, a display 360, and a storage device 370. The support mount 310, the magnet 320, the optical instrument 330, the processing circuitry 340, the input instrument 350, the display 360, and the storage device 370 are connected via a signal line such as a bus such that signals can be transmitted and received via the signal line.

The support mount 310 is a support mechanism for detachably supporting the test cartridge 200A. Attachment and detachment of the test cartridge 200A to and from the support mount 310 is detected electrically, magnetically, or mechanically.

The magnet 320 applies a magnetic field that moves the magnetic particles introduced into the test cartridge 200A. The magnetic field applied by the magnet 320 is controlled by the measurement control function 341A of the processing circuitry 340. The magnet 320 is an example of a measurement unit.

The optical instrument 330 detects light made incident on the translucent substrate of the test cartridge 200A, propagating through the translucent substrate, and emitted from the translucent substrate. An electric signal denoting an intensity of the detected light refers to a measurement signal reflecting a concentration of the measurement target substance contained in the specimen. The measurement signal is supplied to the processing circuitry 340. The optical instrument 330 is an example of a measurement unit.

The processing circuitry 340 is a processor that functions as a control nerve of the optical measurement device 300. By executing programs stored in the storage device 370, etc., the processing circuitry 340 realizes functions corresponding to the programs, namely, the measurement control function 341A, the calculation function 342, the selection function 343A, the quantification function 344, and the output control function 345. In the present embodiment, a case will be described where the measurement control function 341A, the calculation function 342, the selection function 343A, the quantification function 344, and the output control function 345 are realized by a single physical processor; however, the configuration is not limited thereto. For example, the measurement control function 341A, the calculation function 342, the selection function 343A, the quantification function 344, and the output control function 345 may be realized by configuring the processing circuitry of a plurality of independent processors that perform the respective programs.

Through realizing the measurement control function 341A, the processing circuitry 340 optically measures the measurement target substance contained in the specimen in accordance with a measurement sequence, and acquires a measurement signal reflecting a concentration of the measurement target substance. Specifically, the processing circuitry 340 controls the magnet 320 and the optical instrument 330 in accordance with the measurement sequence. The magnetic field to be applied includes, specifically, both a magnetic field (hereinafter referred to as a “lower field”) for bringing the magnetic particles close to the translucent substrate and a field (hereinafter referred to as an “upper field”) for keeping the magnetic particles away from the translucent substrate. The processing circuitry 340 repeatedly acquires a measurement signal output from the optical instrument 330 at the time of performance of the optical measurement. The measurement control function 341A is an example of a measurement unit.

The measurement sequence refers to a time-series order of the processes executed for the optical measurement. As control parameters of the measurement sequence related to control of the magnet 320, an application time of an upper or lower field to a complex containing the measurement target substance, an intensity of the upper or lower field, and/or a spontaneous precipitation time of the complex, for example, may be used; however, the configuration is not particularly limited thereto, and various parameters may be suitably used. The spontaneous precipitation time refers to a time during which neither an upper field nor a lower field is applied. Control parameters of the measurement sequence related to control of the optical instrument 330 include an on/off timing of light irradiation by the optical instrument 330.

Through realizing the calculation function 342, the processing circuitry 340 calculates an index value (hereinafter referred to as a “fluctuation index value”) related to a fluctuation of the measurement signal during a first period. Various parameters with a value that reflects the concentration of the measurement target substance may be used as the fluctuation index value. For example, a single parameter or a combination of multiple parameters representing a fluctuation rate of the intensity of the measurement signal, an integrated value of fluctuation rates, or a maximum or minimum value of the intensity of the measurement signal during the first period may be used as the fluctuation index value; moreover, values obtained by various computations based on a combination of multiple parameters may also be used. The first period is a period during which a measurement signal used for calculating the fluctuation index value is acquired, and is a local period during which the behavior of the intensity of the measurement signal typologically changes in accordance with a concentration range of the measurement target substance. The first period may be included in a second period during which a measurement signal to be used for quantification of the measurement target substance is acquired; however, it is specifically desirable that the first period be set to a partial period from a start time of optical measurement of the measurement target substance to an upper-field application time. The first period is a period during which the control parameters of the measurement sequence are determined. Hereinafter, the first period will be referred to as a “fluctuation measurement period”. Note that the second period may be any period during which a measurement signal to be used for quantification of the measurement target substance is acquired, and is arbitrarily set to include part or entirety of a lower-field application time, the spontaneous precipitation time, and an upper-field application time.

Through realizing the selection function 343A, the processing circuitry 340 selects a single measurement sequence to be used in processing during or after the fluctuation measurement period in accordance with a concentration range corresponding to the fluctuation index value of the measurement target substance. With the measurement control function 341A, a measurement signal is acquired using the selected single measurement sequence in processing during or after the fluctuation measurement period. The concentration range refers to a range of concentration degrees of the measurement target substance. Upon determining, based on a comparison with a threshold that varies according to the fluctuation index value and the fluctuation measurement period, that the concentration range is a first concentration range, the processing circuitry 340 selects a single measurement sequence configured of measurement parameters suitable for quantification of the first concentration range. On the other hand, upon determining that the concentration range is a second concentration range lower than the first concentration range, the processing circuitry 340 selects a single measurement sequence configured of measurement parameters suitable for quantification of the second concentration range. Note that the threshold may be set, through experiments, predictive calculations, etc., to any value that varies according to the fluctuation index value and the fluctuation measurement period; specifically, the threshold is calculated by performing statistical processing in consideration of parameters that affect the reaction, such as the effect of the specimen, the reaction temperature, the subtype of the measurement target substance, etc.

Through realizing the quantification function 344, the processing circuitry 340 quantifies the measurement target substance based on the measurement signal acquired during the second period. Specifically, the processing circuitry 340 calculates, as quantitative values for test items, a concentration value of the measurement target substance based on an intensity of the measurement signal, and a determination result related to whether or not the measurement target substance is positive or negative based on the concentration value. More specifically, the processing circuitry 340 quantifies the measurement target substance for test items based on the measurement signal acquired during the second period and a calibration line corresponding to the measurement target substance. The calibration line may be stored in advance in the storage device 370 according to the type of the substance, or may be retrieved from the outside using various media and means such as one-dimensional and two-dimensional codes and portable storage media such as a flash memory, a CD-ROM, a DVD, and a magnetic medium.

Through realizing the output control function 345, the processing circuitry 340 outputs a variety of information via an output interface such as the display 360. For example, the processing circuitry 340 displays, on the display 360, the measurement sequence selected by the selection function 343A, the quantitative values for various test items obtained with the quantification function 344, and the like.

The input instrument 350 accepts various input operations from an operator, and converts the accepted input operations into operation signals. The operation signals are supplied to the processing circuitry 340. As the input instrument 350, for example, a physical switch, a touch panel, a touch pad, a joystick, a keyboard, etc. may be employed. As the input instrument 350, a speech input device configured to recognize an utterance of an operator perceived by a microphone and convert it into an operation signal may be used.

The display 360 displays, with the output control function 345, various types of information. As the display 360, for example, a liquid-crystal display (LCD), a cathode-ray tube (CRT) display, an organic electroluminescence display (OELD), a plasma display, or any other display may be suitably used. Moreover, the display 360 may be a projector.

The storage device 370 is a storage device configured to store a variety of information, such as a read-only memory (ROM), a random-access memory (RAM), a hard disk drive (HDD), a solid-state drive (SSD), and an integrated-circuit storage device. Moreover, the storage device 370 may be a drive device or a recognition device configured to read and write a variety of information from and to a medium such as a one-dimensional or two-dimensional barcode and a portable storage media such as a flash memory, a CD-ROM, a DVD, or a magnetic medium. Furthermore, the storage device 370 may include a communication device or a communication function for retrieving information from a network or via a wireless communication. Note that the storage device 370 is not necessarily realized by a single storage device. For example, the storage device 370 may be configured of a plurality of storage devices, or a given combination of one or more storage devices and one or more of the above-described drive devices, recognition devices, and/or communication devices. The storage device 370 stores one or more programs, etc. according to the present embodiment. Such programs may be stored in advance in, for example, the storage device 370. Moreover, such programs may be stored in a non-transitory storage medium and distributed, read from the non-transitory storage medium, and installed onto the storage device 370. Furthermore, the control programs may be, for example, downloaded from a network, and installed in the storage device 370.

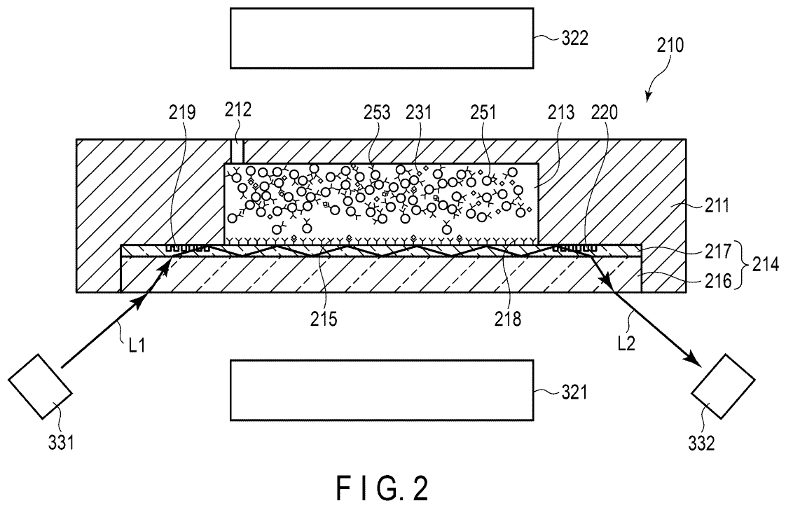

FIG. 2 is a diagram showing a principle of optical measurement of the optical measurement device 300. FIG. 2 shows a cross section of the optical measurement device 300 in which a test cartridge 200A is mounted on a support mount 310 (not illustrated in FIG. 2).

The test cartridge 200A includes a housing 211. The housing 211 is formed, for example, of a resin such as acrylonitrile butadiene styrene (ABS) in an approximately rectangular parallelepiped shape. The housing 211 may be colored in black for the purpose of shielding light. A drop hole 212 is formed in the surface of the housing 211. The drop hole 212 communicates with a reaction tank 213 provided inside the housing 211 via a flow channel.

A translucent substrate 214 is provided on a back surface of the housing 211. The translucent substrate 214 is an example of a measurement unit. The translucent substrate 214 has translucency, and is a substrate on which the first substance 215 that specifically binds to a measurement target substance 231 is fixed. Specifically, the translucent substrate 214 includes a basal portion 216 that has translucency. The basal portion 216 is formed of, for example, alkali-free glass. An optical waveguide 217 is formed on a surface of the basal portion 216 on a side of the reaction tank 213.

As the optical waveguide 217, for example, a planar optical waveguide is used. The optical waveguide 217 can be formed of, for example, a thermosetting resin such as a phenolic resin, an epoxy resin, or an acrylic resin, or can be also formed of a photo-curable resin or alkali-free glass. It is preferable that the optical waveguide 217 have transmittance of predetermined light, and is, for example, a resin, etc. having a higher reflectivity than the basal portion 216.

A part of a surface of the optical waveguide 217 forms a detection surface (sensing area) 218, and forms a bottom surface of the reaction tank 213. A first substance 215 that specifically binds to the measurement target substance 231 is immobilized on the detection surface 218. The first substance 215 is immobilized through, for example, a hydrophobic interaction or chemical binding at a surface of the optical waveguide 217. The detection surface 218 refers to a region where near-field light (evanescent light), which occurs at the surface of the optical waveguide 217, can occur.

A test solution is introduced into the reaction tank 213 via the drop hole 212. As described above, a second substance 253, which specifically binds to the measurement target substance, is bound to the magnetic particles 251. Moreover, the first substance 215, which specifically binds to the measurement target substance 231, is fixed on the detection surface 218. It is assumed that the measurement target substance 231 is an antigen, that the second substance 253 is an antibody (a secondary antibody), and that the first substance 215 is an antibody (a primary antibody).

A grating 219 for incidence and a grating 220 for reflection are provided at both end portions on the surface of the basal portion 216. The grating 219 has a structure of reflecting (refracting) light, and is arranged at a position where light is made incident on the optical waveguide 217. The grating 220 has a structure of reflecting (refracting) light, and is arranged at a position where the light propagating through the optical waveguide 217 is reflected to the outside.

The light source 331 and the photodetector 332 are mounted on the optical measurement device 300 as an optical instrument 330. The light source 331 irradiates the translucent substrate 214 with a light beam L1 under the control of the processing circuitry 340. As the light source 331, a laser diode, a light-emitting diode, etc. may be used. The light beam L1 may be approximately collimated by a separately added lens, etc. The photodetector 332 detects a light beam L2 emitted from the translucent substrate 214. It is preferable that a photodiode be used as the photodetector 332. The photodetector 332 generates a measurement signal denoting an intensity of the detected light beam L2. The optical instrument 330 is an example of the measurement unit.

As shown in FIG. 2, the magnet 320 is provided inside the optical measurement device 300. As shown in FIG. 2, the magnet 320 includes a lower-field magnet 321 and an upper-field magnet 322 so as to sandwich the test cartridge 200A in between. The lower-field magnet 321 and the upper-field magnet 322 are realized by a permanent magnet or an electromagnet. The lower-field magnet 321 applies a lower field for bringing the magnetic particles 251 close to the detection surface 218 under the control of the processing circuitry 340. The upper-field magnet 322 applies an upper field for keeping the magnetic particles 251 away from the detection surface 218 under the control of the processing circuitry 340. The magnet 320 is an example of a measurement unit.

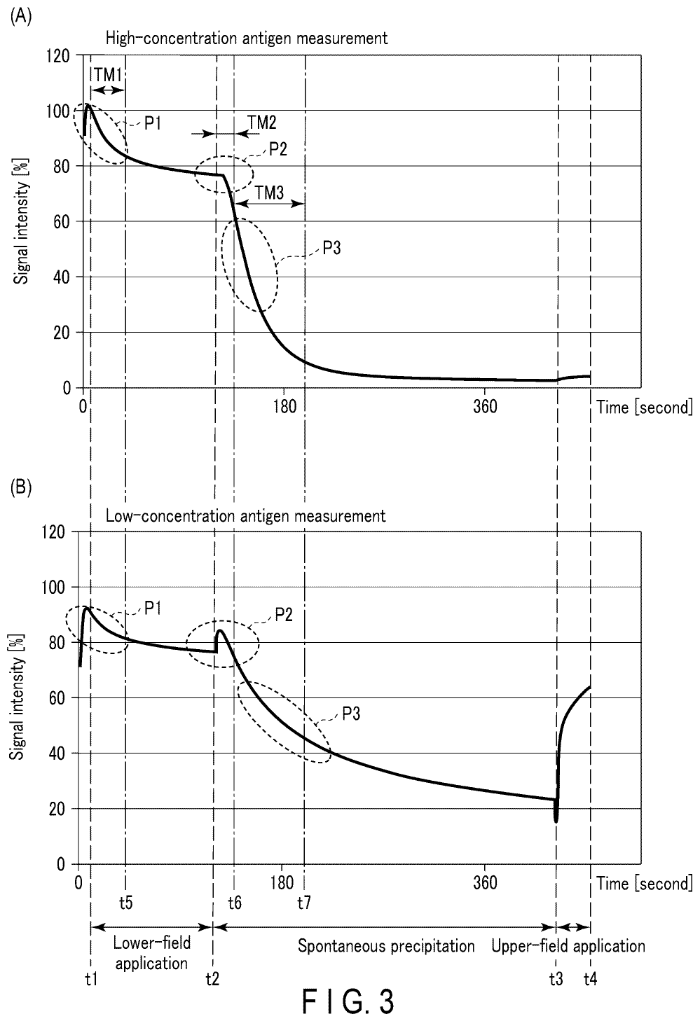

FIG. 3 is a diagram showing temporal changes of an intensity of a measurement signal by comparison between a case of measurement of a high-concentration antigen and a case of measurement of a low-concentration antigen. FIG. 3(A) is a graph showing temporal changes of the intensity of the measurement signal in the case of measurement of a high-concentration antigen, and FIG. 3(B) is a graph showing temporal changes of the intensity of the measurement signal in the case of measurement of a low-concentration antigen. The vertical axis in each graph denotes the intensity [%] of the measurement signal, and the lateral axis denotes time [second]. The “0 seconds” refer to a start time of optical measurement. It is assumed that, at the start time of optical measurement, a test solution is introduced into the reaction tank 213. In FIG. 3, “high concentration” refers to a concentration higher than a reference concentration, and “low concentration” refers to a concentration lower than the reference concentration. The “reference concentration” refers to an upper-limit concentration with a preferable quantitative precision. The “low concentration” refers to, in other words, a non-high concentration.

Referring to FIGS. 3(A) and (B), a standard measurement sequence according to the present embodiment will be described. In optical measurement, the light source 331 allows a light beam to be made incident onto a translucent substrate 214 in accordance with an instruction from the processing circuitry 340. The light beam made incident on the translucent substrate 214 passes through the basal portion 216, is reflected or refracted by the grating 219, and is made incident onto and propagates through the optical waveguide 217. Thereafter, the light beam is reflected or refracted by the grating 220, and emitted from the translucent substrate 214. The photodetector 332 detects the emitted light beam, and outputs a measurement signal denoting an intensity of the detected light beam. The output measurement signal is supplied to the processing circuitry 340. During performance of the optical measurement, the measurement signal is repeatedly acquired.

If the reaction tank 213 is empty, the light propagating through the optical waveguide 217 is not totally reflected, and evanescent light is generated on the detection surface 218. In this case, the signal intensity of the measurement signal takes a low value compared to the case of total reflection. As time passes from the start time (0 seconds) of optical measurement, the test solution dropped into the test cartridge 200A flows into the reaction tank 213. With the detection surface 218 immersed in the test solution, the light beam propagating through the optical waveguide 217 is totally reflected. That is, the signal intensity of the measurement signal increases as time passes from the start time (0 seconds).

As shown in FIGS. 3 (A) and (B), the magnet 320 starts applying a lower field in accordance with an instruction from the processing circuitry 340 at time t1 after the start time of optical measurement. At time t2 after passage of a predetermined period (hereinafter referred to as a “lower-field application period”) from the time t1, the magnet 320 stops applying a lower field in accordance with an instruction from the processing circuitry 340. Through the application of the lower field, the magnetic particles are attracted to the detection surface 218. The magnetic particles are sequentially attracted to the detection surface 218, and thus form a large number of horn-like bundles aligned along the magnetic field line of the lower field on the detection surface 218.

A no-field state is maintained from the time t2 until a time t3 after passage of a predetermined period (hereinafter referred to as a “spontaneous precipitation period”). During the spontaneous precipitation period, the horn-like bundles of magnetic particles aligned on the detection surface 218 are unraveled and settle onto the detection surface 218. At this point in time, the intensity of leakage light from the detection surface 218 increases, causing the signal intensity of the measurement signal to decrease.

At time t3, the magnet 320 starts applying an upper field in accordance with an instruction from the processing circuitry 340. At time t4 after passage of a predetermined period (hereinafter referred to as an “upper-field application period”) from the time t3, the magnet 320 stops applying an upper field in accordance with an instruction from the processing circuitry 340. Through the application of the upper field, the magnetic particles settled on the detection surface 218 are repelled from the detection surface 218. In accordance therewith, the signal intensity of the measurement signal increases. On the other hand, the magnetic particles bound to the antigen are bound to the antibody fixed to the detection surface 218, and are not repelled from the detection surface 218 even by the application of the upper field and remain thereon. Accordingly, if an antigen does not exist in the specimen, the signal intensity of the optical detection signal reverts to an initial state; however, if an antigen does exist in the specimen, the signal intensity of the measurement signal does not revert to the initial state, and remains at a value lower than the initial state.

The signal intensity of the measurement signal acquired in the upper-field application period reflects a concentration of the measurement target substance. Thus, quantification of the measurement target substance for test items is performed with the quantification function 344 using some or all of the measurement signals during the upper-field application period. The upper-field application period in a standard measurement sequence is set, for example, within a period of 400 to 500 seconds from the measurement start time. Note that the measurement signal required for quantification is not limited to some or all of the measurement signals during the upper-field application period, and other measurement signals may be used; furthermore, quantification may be performed based on a result of computation of a combination of multiple measurement signals.

The behavior of the signal intensity of the measurement signal differs between the high-concentration antigen and the low-concentration antigen, as shown in FIGS. 3 (A) and (B). In the first example, the intensity of the measurement signal reaches its peak during a period P1 immediately after the time t1 of starting of the lower-field application, with the fall from the peak being sharp in the high-concentration case but being more gradual in the low-concentration case. Thus, a concentration range of the measurement target substance is determined based on the fluctuation index value of the intensity of the measurement signal during a fluctuation measurement period TM1 corresponding to the period P1. As the fluctuation index value, a fluctuation rate of the intensity of the measurement signal, an integrated value of fluctuation rates, or the like during the fluctuation measurement period TM1 is used. The concentration range is typically divided into a range of high concentrations (hereinafter referred to as a “high-concentration range”) and a range of low concentrations (hereinafter referred to as a “low-concentration range”). If the fluctuation index value is larger than the threshold A, the concentration range is determined to be the high-concentration range, and if the fluctuation index value is smaller than a first threshold, the concentration range is determined to be the low-concentration range. The threshold A may be arbitrarily determined by experiments, predictive calculations, etc.

In the case where the measurement target substance is not contained at a high concentration, the fluctuation measurement period TM1 is set to a local period including an arrival time of a peak of the intensity of the measurement signal upon start of the lower-field application. As an example, the period TM1 is set to a local period during a period ranging from the start time of optical measurement, through the time t1, to the time t2, and including an empirical arrival time of a peak. A time width of the period TM1 can be arbitrarily set.

In the second example, the intensity of the measurement signal jumps up in the low-concentration case during a period P2 immediately after the time t2 when the lower-field application is stopped; however, the intensity of the measurement signal does not jump up in the high-concentration case. Thus, a concentration range of the measurement target substance is determined based on the fluctuation index value of the intensity of the measurement signal during a fluctuation measurement period TM2 corresponding to the period P2. As the fluctuation index value, a fluctuation rate of the intensity of the measurement signal, an integrated value of fluctuation rates, or the like during the fluctuation measurement period TM2 is used. If the fluctuation index value is larger than the threshold B, the concentration range is determined to be the low-concentration range, and if the fluctuation index value is smaller than the threshold B, the concentration range is determined to be the high-concentration range. The threshold B may be arbitrarily determined by experiments, predictive calculations, etc.

In the case where the measurement target substance is not contained at a high concentration, the fluctuation measurement period TM2 is set to a local period including an arrival time of a jump up in the intensity of the measurement signal upon termination of the lower-field application. As an example, the fluctuation measurement period TM2 is set to a period ranging from the time t2 to a time when a jump up can be detected. The time when a jump up can be detected is set to be any time point when a jump up can be detected based on the fluctuation index value, and may be either earlier than or later than an arrival time of a jump up peak from the time t2.

In the third example, the intensity of the measurement signal falls sharply during a falling period P3 of the intensity of the measurement signal from the time t2 of stopping of the lower-field application in the high-concentration case, but is more gradual in the low-concentration case. Thus, a concentration range of the measurement target substance is determined based on the fluctuation index value of the intensity of the measurement signal during a fluctuation measurement period TM3 corresponding to the falling period P3. As the fluctuation index value, a fluctuation rate of the intensity of the measurement signal, an integrated value of fluctuation rates, or the like during the fluctuation measurement period TM3 is used. If the fluctuation index value is larger than a threshold C, the concentration range is determined to be the high-concentration range, and if the fluctuation index value is smaller than the threshold C, the concentration range is determined to be the low-concentration range. The threshold C may be arbitrarily determined by experiments, predictive calculations, etc.

The fluctuation measurement period TM3 is set to a local period during which the intensity of the measurement signal decreases upon termination of the lower-field application. As an example, the fluctuation measurement period TM3 is set to a period ranging from a time when a jump up can be detected to an estimated convergence time. The estimated convergence time is set to a time point when a decrease in the intensity of the measurement signal is empirically estimated to converge. The estimated convergence time is set earlier than the time t3 of starting of the upper-field application.

Note that the fluctuation measurement period is not limited to the fluctuation measurement periods TM1, TM2, and TM3 in the above-described example, and may be set to a given measurement period. Moreover, the number of fluctuation measurement periods may be either one or more than one. Furthermore, it is desirable that the fluctuation measurement period be selected from TM1, TM2, or TM3; however, considering that calculating a concentration range during the fluctuation measurement period TM1, which is an initial phase of measurement, allows the subsequent measurement sequence conditions to be diversified, it is most desirable, where possible, that the concentration range be calculated by setting an initial stage of measurement as the fluctuation measurement period.

Next, a procedure for performing optical measurement with the immunoassay system 100 according to the first embodiment will be described.

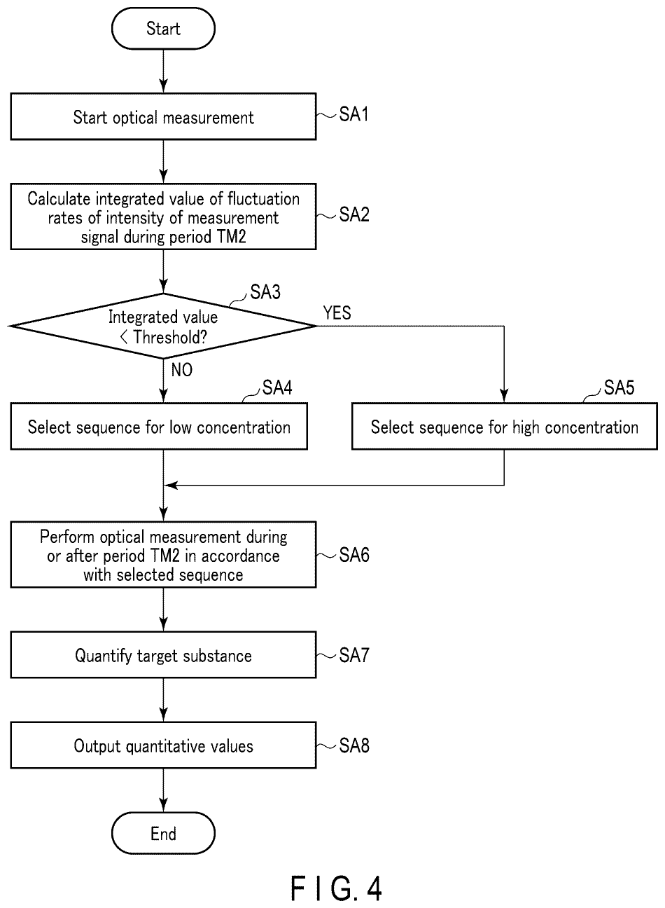

FIG. 4 is a diagram showing a procedure for performing optical measurement with the immunoassay system 100 according to the first embodiment. It is assumed that, at a start time of step SA1, a specimen treatment liquid is introduced into the reaction tank 213 of the test cartridge 200A. It is also assumed that a first period used for calculation of a fluctuation index value is the fluctuation measurement period TM2 shown in FIG. 3, and that the fluctuation index value is an integrated value of fluctuation rates of the intensity of the measurement signal over the fluctuation measurement period TM2.

First, the processing circuitry 340 starts optical measurement with the measurement control function 341A (step SA1). In the optical measurement at step SA1, the processing circuitry 340 repeatedly acquires, with the measurement control function 341A, a measurement signal that reflects a concentration of the measurement target substance contained in the specimen treatment liquid from the optical instrument 330. At the start time of optical measurement, it suffices that optical measurement is performed in accordance with a standard measurement sequence.

After step SA1, the processing circuitry 340 calculates, with the calculation function 342, an integrated value of fluctuation rates of the measurement value during the fluctuation measurement period TM2 (step SA2). Specifically, at step SA2, the processing circuitry 340 calculates a difference in intensity between measurement signals at two neighboring measurement points in a period TM2. The calculated difference refers to a gradient of the intensity of the measurement signal, in other words, a fluctuation rate. In this manner, the processing circuitry 340 calculates a fluctuation rate at each measurement point in the period TM2. Subsequently, the processing circuitry 340 calculates a total sum of fluctuation rates respectively corresponding to the measurement points obtained during the period TM2 as an integrated value.

After step SA2, the processing circuitry 340 determines, with the selection function 343A, whether or not the integrated value calculated at step SA2 is smaller than a threshold (step SA3).

If it is determined at step SA3 that the integrated value is not smaller than the threshold (step SA3: NO), the processing circuitry 340 selects, with the selection function 343A, a measurement sequence for low concentration (step SA4). The measurement sequence for low concentration refers to a measurement sequence with a measurement time longer than a measurement time of a standard measurement sequence suitable for quantification of the measurement target substance in the low-concentration range. As an example, the measurement time of the standard measurement sequence is set on the order of 450 seconds; however, it is preferable that the measurement sequence for low concentration be set on the order of approximately 600 seconds. An extension of the measurement time should be realized by, for example, extending the spontaneous precipitation time compared to the standard measurement sequence. It is assumed that a time width of the upper-field application period is equivalent to that of the standard measurement sequence.

If it is determined at step SA3 that the integrated value is smaller than the threshold (step SA3: YES), the processing circuitry 340 selects, with the selection function 343A, a measurement sequence for high concentration (step SA5). The measurement sequence for high concentration refers to a measurement sequence shorter than a measurement time for a standard measurement sequence suitable for quantification of the measurement target substance in the high-concentration range. It is preferable that the measurement sequence for low concentration be set on the order of approximately 180 seconds. It is preferable that the measurement time be shortened by, for example, shortening the spontaneous precipitation time. It is assumed that a time width of the upper-field application period is equivalent to that of the standard measurement sequence.

After step SA4 or SA5, the processing circuitry 340 performs, with the measurement control function 341A, an optical measurement during or after the period TM2 in accordance with the measurement sequence selected at step SA4 or SA5 (step SA6). That is, if the measurement sequence for high concentration is selected, the spontaneous precipitation time is shortened, thus accelerating the start time of the upper-field application; on the other hand, if the measurement sequence for low concentration is selected, the spontaneous precipitation time is extended, thus delaying the start time of the upper-field application.

After step SA6, the processing circuitry 340 quantifies, with the quantification function 344, the measurement target substance (step SA7). At step SA7, the processing circuitry 340 calculates, as quantitative values, a concentration value of the measurement target substance and a determination result based on the concentration value as to, for example, whether the measurement target substance is positive or negative, based on a signal intensity of the measurement signal acquired during a quantitative measurement period set during or after the fluctuation measurement period TM2. Specifically, the processing circuitry 340 selects a calibration line corresponding to the measurement target substance from a plurality of calibration lines respectively corresponding to a plurality of substances stored in the storage device 370. The calibration line refers to a straight or curved line denoting a relationship between a concentration of the substance whose concentration value is known and a calculation measurement value of the intensity of the measurement signal. The processing circuitry 340 calculates a concentration value of the measurement target substance based on a comparison between the selected calibration line and the signal intensity of the measurement signal acquired during the upper-field application time. For example, the processing circuitry 340 determines that the measurement target substance is positive if the calculated concentration value exceeds a threshold, and determines that the measurement target substance is negative if the calculated concentration value falls below the threshold.

After step SA7, the processing circuitry 340 outputs, with the output control function 345, the quantitative values obtained at step SA7 (step SA8). For example, the processing circuitry 340 displays, on the display 360, the quantitative values in a predetermined layout.

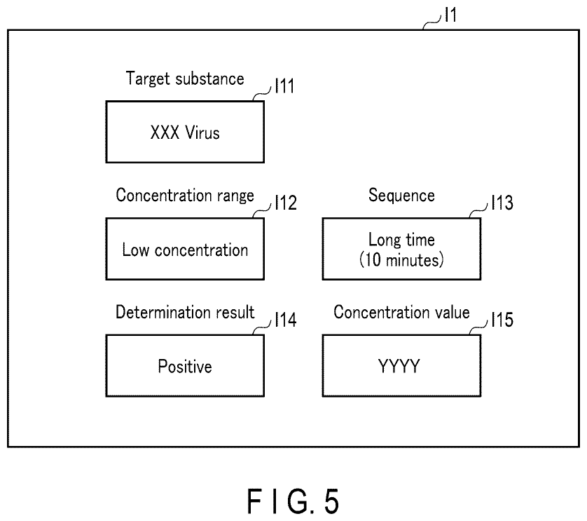

FIG. 5 is a diagram showing an example of a display screen I1 for the quantitative values according to the first embodiment. As shown in FIG. 5, the display screen I1 includes a display column I11 for the measurement target substance, a display column I12 for the concentration range, a display column I13 for the measurement sequence, a display column I14 for the determination result, and a display column I15 for the concentration value. In the display column I11, a name, etc. of the measurement target substance such as “XXX Virus” is displayed. In the display column I12, a character string denoting a type of the concentration range of the measurement target substance determined at step SA3, such as “low concentration”, is displayed. In the display column I13, a character string denoting a type of the measurement sequence selected at step SA4 or SA5, such as “long time (10 min)”, is displayed. In the display column I14, a character string denoting a positive/negative determination result obtained at step SA7, such as “positive”, is displayed. In the display column I15, a numerical value denoting the concentration value of the measurement target substance obtained at step SA7, such as “YYYY”, is displayed. Through displaying of the concentration range and the type of the measurement sequence together with the quantitative values such as the positive/negative determination result and the concentration value, it is possible for the user to grasp the measurement sequence that has been executed, and to grasp the reason why the measurement sequence has been selected. Note that some of the display columns I11 to I15 may be omitted, and other information may be displayed.

After step SA7, the procedure for the optical measurement shown in FIG. 4 ends.

As described above, according to the first embodiment, the immunoassay system 100 determines, in a simplified manner, a concentration range of a measurement target substance based on a measurement signal acquired during a fluctuation measurement period prior to a quantitative measurement period, selects a measurement sequence corresponding to the concentration range, and performs an optical measurement during or after the fluctuation measurement period using the selected measurement sequence. Thereby, the measurement sequence corresponding to the concentration range of the measurement target substance is executed, thus enlarging the dynamic range related to the concentration and improving the precision of measurement.

### Second Embodiment

An immunoassay system 100 according to a second embodiment selects a measurement channel according to a concentration range of a measurement target substance. Hereinafter, the immunoassay system 100 according to the second embodiment will be described. In the description that follows, structural components having substantially the same functions as those of the first embodiment will be assigned identical reference symbols, and a repetitive description will be given only where necessary.

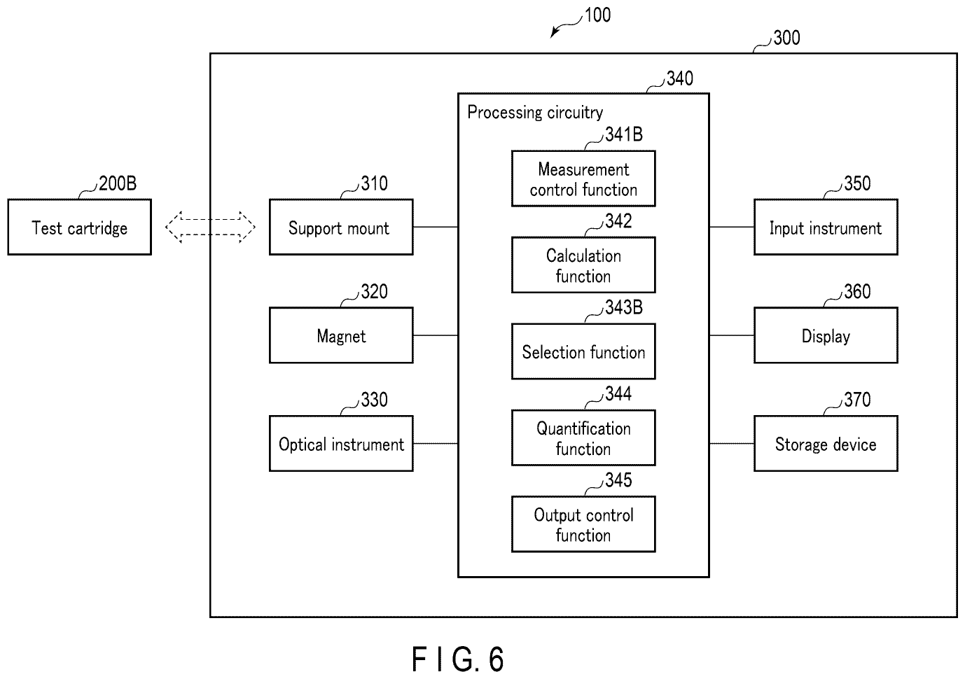

FIG. 6 is a diagram showing a configuration example of the immunoassay system 100 according to the second embodiment. As shown in FIG. 6, the immunoassay system 100 according to the second embodiment includes a test cartridge 200B instead of the test cartridge 200A. For a single reaction tank of the test cartridge 200B, a plurality of combinations of a detection surface 218 and an optical instrument 330 are prepared. Each combination of the detection surface 218 and the optical instrument 330 configures a measurement channel. The test cartridge 200B includes a plurality of measurement channels in which a primary antibody immobilized on the detection surface 218 exhibits different reagent properties. The reagent properties include a rate of reaction and/or an efficiency of reaction between the primary antibody and the measurement target substance. A measurement signal obtained at each measurement channel is supplied to the processing circuitry 340.

As shown in FIG. 6, the processing circuitry 340 realizes a measurement control function 341B and a selection function 343B, as well as a calculation function 342, a quantification function 344, and an output control function 345.

Through realizing the measurement control function 341B, the processing circuitry 340 optically measures a measurement target substance contained in a specimen at some or all of the measurement channels in which the immobilized primary antibody exhibits different reagent properties, and acquires a measurement signal reflecting a concentration of the measurement target substance. Specifically, the processing circuitry 340 controls the magnet 320 and the optical instrument 330 in accordance with the measurement sequence. In the measurement control function 341B, a standard single measurement sequence is used as the measurement sequence. The processing circuitry 340 repeatedly acquires measurement signals output from the optical instrument 330 at the time of performance of an optical measurement using some or all of the measurement channels that are mounted. After selection of a measurement channel with the selection function 343B, the selected measurement channel is used. Prior to selection of the measurement channel with the selection function 343B, a given measurement channel is used. The measurement control function 341B is an example of a measurement unit.

Through realizing the selection function 343B, the processing circuitry 340 selects, from among of the plurality of measurement channels, a single measurement channel to be used in processing during or after the fluctuation measurement period in accordance with a concentration range corresponding to a fluctuation index value of the measurement target substance. With the measurement control function 341B, a measurement signal is acquired using the selected single measurement channel in processing during or after the fluctuation measurement period. Upon determining, based on a comparison with a threshold that varies according to the fluctuation index value and the fluctuation measurement period, that the concentration range is a first concentration range, the processing circuitry 340 selects a single measurement channel having reagent properties suitable for quantification of the first concentration range. On the other hand, upon determining that the concentration range is a second concentration range lower than the first concentration range, the processing circuitry 340 selects a single measurement channel having reagent properties suitable for quantification of the second concentration range.

The expression “select a measurement channel” refers to using a measurement signal output from the measurement channel in subsequent processing. That is, it encompasses not only a case where the selected measurement channel is driven and the non-selected measurement channels are stopped, but also a case where both the selected measurement channel and the non-selected measurement channels are driven. In the latter case, measurement signals output from both of the selected and non-selected measurement channels are supplied to the processing circuitry 340, and only the measurement signal from the selected measurement channel is used in the subsequent processing.

Next, a procedure for optical measurement with the immunoassay system 100 according to the second embodiment will be described.

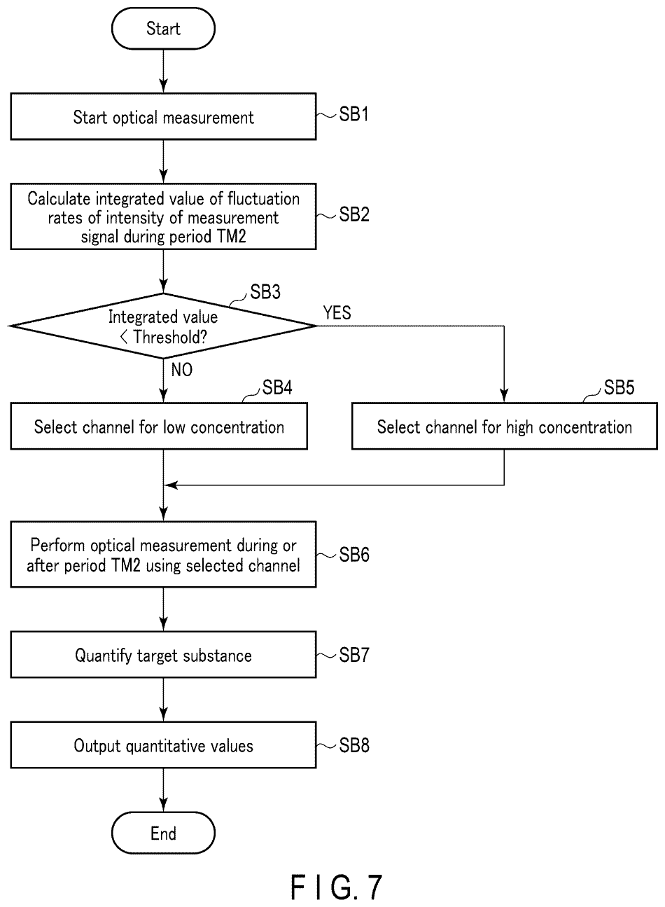

FIG. 7 is a diagram showing a procedure for optical measurement with the immunoassay system 100 according to the second embodiment. It is assumed that, at a start time of step SB1, a specimen treatment liquid is introduced into a reaction tank of the test cartridge 200B. It is also assumed that a first period used for calculation of the fluctuation index value is a period TM2 shown in FIG. 3, and a fluctuation index value is an integrated value of fluctuation rates of the intensity of the measurement signal over the period TM2. It is also assumed that two types of measurement channels, namely, a measurement channel for low concentration and a measurement channel for high concentration, are mounted on the immunoassay system 100. The measurement channel for low concentration refers to a measurement channel on which an antibody having reagent properties with high reactivity, suitable for quantification of the measurement target substance in the low-concentration range, is immobilized, compared to the measurement channel for high concentration. The measurement channel for high concentration refers to a measurement channel on which an antibody having reagent properties with low reactivity, suitable for quantification of the measurement target substance in the high-concentration range, is immobilized, compared to the measurement channel for low concentration.

First, the processing circuitry 340 starts optical measurement with the measurement control function 341B (step SB1). In the optical measurement at step SB1, the processing circuitry 340 repeatedly acquires, with the measurement control function 341B, a measurement signal that reflects the concentration of the measurement target substance contained in the specimen treatment liquid from the optical instrument 330. During the period from start of the optical measurement to termination of the fluctuation measurement period, either one of or both of the measurement channel for low concentration and the measurement channel for high concentration may be used.

After step SB1, the processing circuitry 340 calculates, with the calculation function 342, an integrated value of fluctuation rates of the measurement value during the fluctuation measurement period TM2 (step SB2). A method of calculating the integrated value of the fluctuation rates is similar to that at step SA2.

After step SB2, the processing circuitry 340 determines, with the selection function 343B, whether or not the integrated value calculated at step SB2 is smaller than a threshold (step SB3).

If it is determined at step SB3 that the integrated value is not smaller than the threshold (step SB3: NO), the processing circuitry 340 selects, with the selection function 343B, a measurement channel for low concentration (step SB4). If it is determined at step SB3 that the integrated value is smaller than the threshold (step SB3: YES), the processing circuitry 340 selects, with the selection function 343B, a measurement channel for high concentration (step SB5).

After step SB4 or SB5, the processing circuitry 340 performs, with the measurement control function 341B, an optical measurement during or after the fluctuation measurement period TM2 using the measurement channel selected at step SB4 or SB5 (step SB6).

After step SB6, the processing circuitry 340 quantifies, with the quantification function 344, the measurement target substance (step SB7). At step SB7, from the measurement channel selected at step SB6, the processing circuitry 340 calculates, as quantitative values, a concentration value of the measurement target substance and a determination result based on the concentration value as to, for example, whether the measurement target substance is positive or negative, based on a signal intensity of the measurement signal acquired during the quantitative measurement period set during or after the fluctuation measurement period TM2. A method of calculating the quantitative values is similar to that at step SA7.

After step SB7, the processing circuitry 340 outputs, with the output control function 345, the quantitative values obtained at step SB7 (step SB8). For example, the processing circuitry 340 displays, on the display 360, the quantitative values in a predetermined layout.

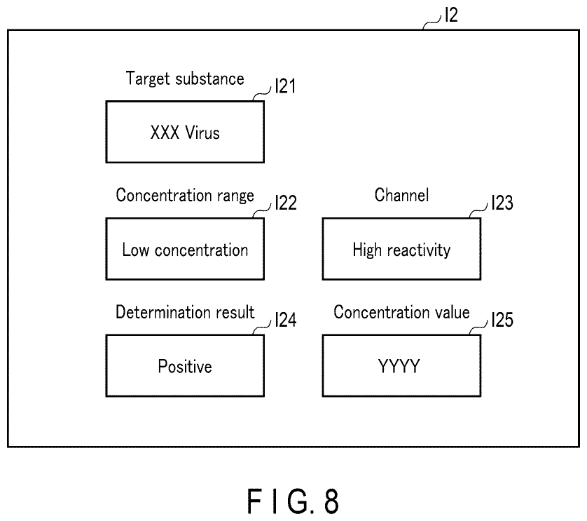

FIG. 8 is a diagram showing an example of a display screen 12 for the quantitative values according to the second embodiment. As shown in FIG. 8, the display screen 12 includes a display column I21 for the measurement target substance, a display column I22 for the concentration range, a display column I23 for the measurement channel, a display column I24 for the determination result, and a display column I25 for the concentration value. In the display column I21, a name, etc. of the measurement target substance such as “XXX Virus” is displayed. In the display column I22, a character string denoting a type of the concentration range of the measurement target substance determined at step SB3, such as “low concentration”, is displayed. In the display column I23, a character string denoting a type of the measurement channel selected at step SB4 or SB5, such as “high reactivity”, is displayed. In the display column I24, a character string denoting a positive/negative determination result obtained at step SB7, such as “positive”, is displayed. In the display column I25, a numerical value denoting the concentration value of the measurement target substance obtained at step SB7, such as “YYYY”, is displayed. Through displaying of the concentration range and the type of the measurement channel together with the quantitative values such as the positive/negative determination result and the concentration value, it is possible for the user to grasp the measurement channel that has been used, and to grasp the reason why the measurement channel has been selected. Note that some of the display columns I21 to I25 may be omitted, or other information may be displayed.

After step SB7, the procedure for the optical measurement shown in FIG. 7 ends.

As described above, according to the second embodiment, the immunoassay system 100 determines, in a simplified manner, a concentration range of the measurement target substance based on a measurement signal acquired during a fluctuation measurement period prior to a quantitative measurement period, selects a measurement channel according to the concentration range, and performs an optical measurement during or after the fluctuation measurement period using the selected measurement channel. Thereby, the measurement channel corresponding to the concentration range of the measurement target substance is executed, thus enlarging the dynamic range related to the concentration and improving the precision of measurement.

### Third Embodiment

An immunoassay system 100 according to a third embodiment selects a measurement sequence and a measurement channel according to a concentration range of a measurement target substance. Hereinafter, the immunoassay system 100 according to the third embodiment will be described. In the description that follows, structural components having substantially the same functions as those of the first and second embodiments will be assigned identical reference symbols, and a repetitive description will be given only where necessary.

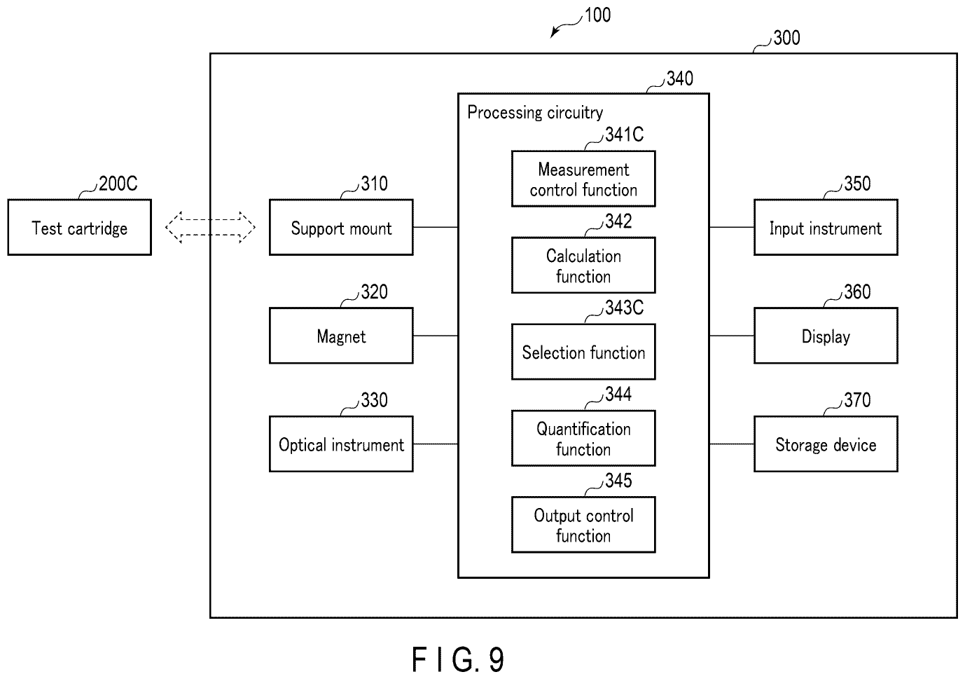

FIG. 9 is a diagram showing a configuration example of the immunoassay system 100 according to the third embodiment. As shown in FIG. 9, the immunoassay system 100 includes a test cartridge 200C. The immunoassay system 100 according to the second embodiment includes the test cartridge 200C instead of the test cartridge 200A. The test cartridge 200C includes, similarly to the test cartridge 200B, a plurality of measurement channels in which the primary antibody immobilized on the detection surface 218 exhibits different reagent properties.

As shown in FIG. 9, the processing circuitry 340 realizes a measurement control function 341C and a selection function 343C, as well as a calculation function 342, a quantification function 344, and an output control function 345.

Through realizing the measurement control function 341C, the processing circuitry 340 optically measures, in accordance with the measurement sequence, a measurement target substance contained in a specimen in some or all of the measurement channels in which the immobilized primary antibody exhibits different reagent properties, and acquires a measurement signal reflecting a concentration of the measurement target substance. Specifically, the processing circuitry 340 controls the magnet 320 and the optical instrument 330 in accordance with the measurement sequence. In the third embodiment, a plurality of measurement sequences are prepared according to a concentration range, similarly to the first embodiment. The processing circuitry 340 repeatedly acquires measurement signals output from the optical instrument 330 at the time of performance of an optical measurement using some or all of the measurement channels that are mounted, similarly to the second embodiment. After selection of a measurement channel and a measurement sequence with the selection function 343C, the selected measurement channel and the selected measurement sequence are used. Prior to selection of the measurement channel with the selection function 343C, a given measurement channel is used, similarly to the second embodiment, and prior to selection of the measurement sequence with the selection function 343C, a standard measurement sequence is used, similarly to the first embodiment. The measurement control function 341C is an example of a measurement unit.

Through realizing the selection function 343C, the processing circuitry 340 selects a single measurement channel and a single measurement sequence to be used in a second period during or after a first period in accordance with a concentration range corresponding to a fluctuation index value of the measurement target substance. With the measurement control function 341C, an optical measurement on a measurement target substance is performed in the second period using the selected single measurement channel and the selected single measurement sequence. The processing circuitry 340 selects a single measurement channel and a single measurement sequence based on a comparison with a threshold that varies according to the fluctuation index value and the fluctuation measurement period. Thresholds related to the third embodiment include a threshold for selection of the measurement sequence and a threshold for selection of the measurement channel. Assuming, for example, that each of the measurement sequence and the measurement channel is divided into those for high concentration and those for low concentration, it follows that there will be four combinations of the measurement sequence and the measurement channel that can be selected, namely, the measurement sequence for high concentration and the measurement channel for high concentration, the measurement sequence for high concentration and the measurement channel for low concentration, the measurement sequence for low concentration and the measurement channel for high concentration, and the measurement sequence for low concentration and the measurement channel for low concentration.

Next, a procedure for optical measurement with the immunoassay system 100 according to the third embodiment will be described.

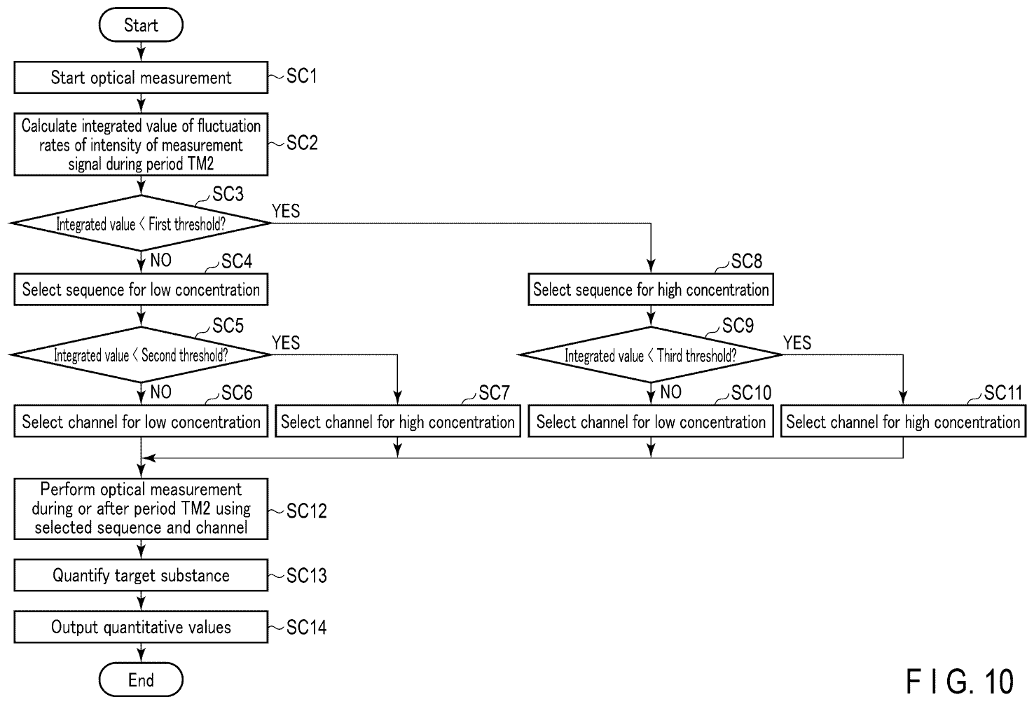

FIG. 10 is a diagram showing a procedure for optical measurement with the immunoassay system 100 according to the third embodiment. It is assumed that, at a start time of step SB1, a specimen treatment liquid is introduced into a reaction tank of the test cartridge 200B. It is also assumed that a first period used for calculation of the fluctuation index value is a period TM2 shown in FIG. 3, and a fluctuation index value is an integrated value of fluctuation rates of the intensity of the measurement signal over the period TM2. It is also assumed that two types of measurement channels, namely, a measurement channel for low concentration and a measurement channel for high concentration, are mounted on the immunoassay system 100.

First, the processing circuitry 340 starts optical measurement with the measurement control function 341C (step SC1). In the optical measurement at step SC1, the processing circuitry 340 repeatedly acquires, with the measurement control function 341C, a measurement signal that reflects a concentration of the measurement target substance contained in the specimen treatment liquid from the optical instrument 330. As the measurement channel, either one of or both of the measurement channel for low concentration and the measurement channel for high concentration may be used. In the present embodiment, for convenience, it is assumed that one of the measurement channels (an initial channel) is used. At the start time of optical measurement, it suffices that optical measurement is performed in accordance with a standard measurement sequence.

After step SC1, the processing circuitry 340 calculates, with the calculation function 342, an integrated value of fluctuation rates of the measurement value during the fluctuation measurement period TM2 (step SC2). A method of calculating the integrated value of the fluctuation rates is similar to that at step SA2.

After step SC2, the processing circuitry 340 determines, with the selection function 343C, whether or not the integrated value calculated at step SC2 is smaller than a first threshold (step SC3).

If it is determined at step SC3 that the integrated value is not smaller than the first threshold (step SC3: NO), the processing circuitry 340 selects, with the selection function 343C, a measurement sequence for low concentration (step SC4).

After step SC4, the processing circuitry 340 determines whether or not the integrated value calculated at step SC2 is smaller than a second threshold (step SC5). If it is determined at step SC5 that the integrated value is not smaller than the second threshold (step SC5: NO), the processing circuitry 340 selects, with the selection function 343C, a measurement channel for low concentration (step SC6). If it is determined at step SC5 that the integrated value is smaller than the threshold (step SC5: YES), the processing circuitry 340 selects, with the selection function 343C, a measurement channel for high concentration (step SC7).

On the other hand, if it is determined at step SC3 that the integrated value is smaller than the first threshold (step SC3: YES), the processing circuitry 340 selects, with the selection function 343C, measurement sequences for high and low concentrations (step SC8). After step SC8, the processing circuitry 340 determines whether or not the integrated value calculated at step SC2 is smaller than a third threshold (step SC9). If it is determined at step SC9 that the integrated value is not smaller than the third threshold (step SC9: NO), the processing circuitry 340 selects, with the selection function 343C, a measurement channel for low concentration (step SC10). If it is determined at step SC9 that the integrated value is smaller than the threshold (step SC9: YES), the processing circuitry 340 selects, with the selection function 343C, a measurement channel for high concentration (step SC11).

After step SC6, SC7, SC10, or SC11, the processing circuitry 340 performs, with the measurement control function 341C, an optical measurement during or after the fluctuation measurement period TM2 using the measurement channel selected at step SC6, SC7, SC10, or SC11 in accordance with the measurement sequence selected at step SC4 or SC9 (step SC12).

After step SC12, the processing circuitry 340 quantifies, with the quantification function 344, the measurement target substance (step SC13). From the measurement channel selected at step SC13, the processing circuitry 340 calculates, as quantitative values, a concentration value of the measurement target substance and a determination result based on the concentration value as to, for example, whether the measurement target substance is positive or negative, based on a signal intensity of the measurement signal acquired during a quantitative measurement period set during or after the fluctuation measurement period TM2. A method of calculating the quantitative values is similar to that at step SA7.

After step SC13, the processing circuitry 340 outputs, with the output control function 345, the quantitative values obtained at step SC13 (step SC14). For example, the processing circuitry 340 displays, on the display 360, the quantitative values in a predetermined layout.

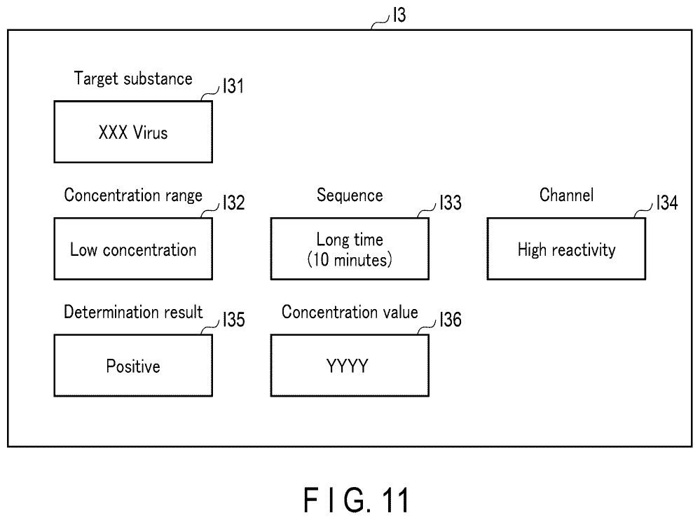

FIG. 11 is a diagram showing an example of a display screen 13 for the quantitative values according to the third embodiment. As shown in FIG. 11, the display screen 13 includes a display column I31 for the measurement target substance, a display column I32 for the concentration range, a display column I33 for the measurement sequence, a display column I34 for the measurement channel, a display column I35 for the determination result, and a display column I36 for the concentration value. In the display column I31, a name, etc. of the measurement target substance such as “XXX Virus” is displayed. In the display column I32, a character string denoting a type of the concentration range of the measurement target substance determined at step SC3, such as “low concentration”, is displayed. In the display column I33, a character string denoting a type of the measurement sequence selected at step SC4 or SC8, such as “long time (10 min)”, is displayed. In the display column I34, a character string denoting a type of a measurement channel selected at step SC6, SC7, SC10, or SC11, such as “high reactivity”, is displayed. In the display column I35, a character string denoting a positive/negative determination result obtained at step SC13, such as “positive”, is displayed. In the display column I36, a numerical value denoting the concentration value of the measurement target substance obtained at step SC13, such as “YYYY”, is displayed. Through displaying of the concentration range, the type of the measurement sequence, and the type of the measurement channel together with the quantitative values such as the positive/negative determination result and the concentration value, it is possible for the user to grasp the measurement sequence and the measurement channel that have been used, and to grasp the reason why the measurement sequence and the measurement channel have been selected. Note that some of the display columns I31 to I36 may be omitted, or other information may be displayed.

After step SC13, the procedure for the optical measurement shown in FIG. 10 ends.

In the above-described embodiment, selection of the measurement channel is performed after selection of the measurement sequence; however, measurement of the measurement sequence may be performed after selection of the measurement channel.

As described above, according to the third embodiment, the immunoassay system 100 determines, in a simplified manner, a concentration range of the measurement target substance based on a measurement signal acquired during a fluctuation measurement period prior to a quantitative measurement period, selects a measurement sequence corresponding to the concentration range, and performs an optical measurement during or after the fluctuation measurement period using the selected measurement sequence. Thereby, the measurement sequence corresponding to the concentration range of the measurement target substance is executed, thus enlarging the dynamic range related to the concentration and improving the precision of measurement.

### Example 1

Using a specimen containing an antigen at a predetermined concentration, a concentration value of the antigen according to each of the first, second, and third embodiments was measured. As a first period, a period TM1 shown in FIG. 3 was used. As the fluctuation index value, an integrated value Sx1 of fluctuation rates of the intensity of the measurement signal was used. Measurement was performed three times.

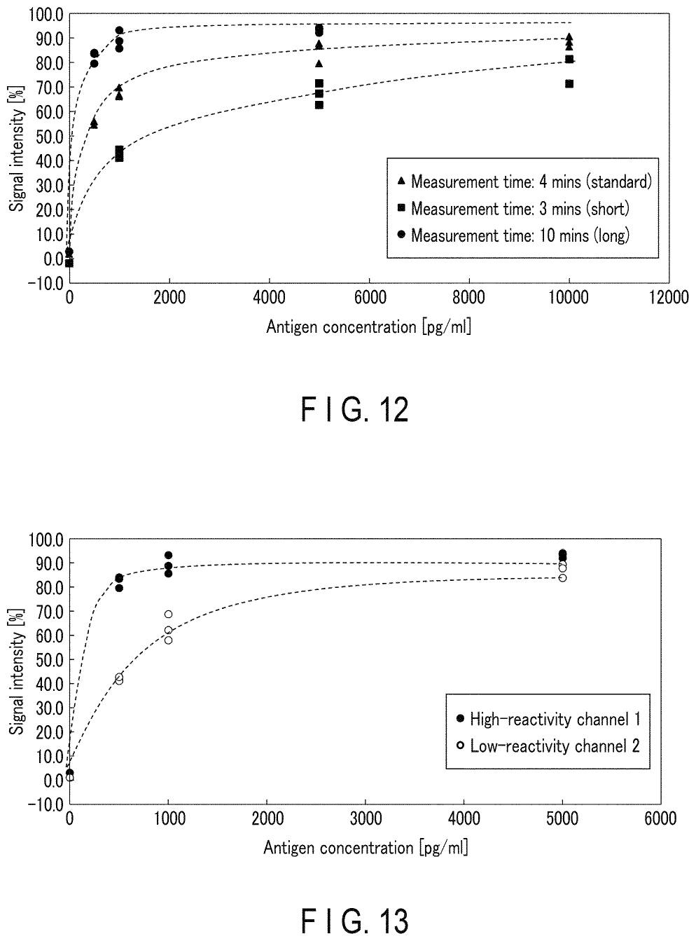

FIG. 12 is a diagram showing a measurable concentration range under each measurement condition. In the graph of FIG. 12, the vertical axis denotes a signal intensity [%] of the measurement signal, and the lateral axis denotes an antigen concentration [pg/ml]. The measurement condition shown in FIG. 12 denotes a combination of a measurement sequence and a measurement channel.

In connection with the first embodiment, if Sx1<threshold, the concentration range of the antigen falls in the low-concentration range. In this case, a measurement sequence with a long measurement time (10 minutes) as denoted by the black circle symbols in FIG. 12 and suitable for quantification of the low-concentration range of the antigen, compared to the 4-minute sequence denoted by the black triangle marks in FIG. 12, was used. It can be seen, from the measurement of a specimen containing an antigen at a concentration higher than 1000 μg/ml, that the signal intensity of the measurement signal approximates its maximum value. On the other hand, if Sx1>threshold, the concentration range of the antigen falls in the high-concentration range, and thus a measurement sequence with a short measurement time (3 minutes) suitable for quantification of the high-concentration range, as denoted by the black square symbols in FIG. 12, was used. In this case, it can be seen that the signal intensity of the measurement signal approximates its maximum value at a concentration equal to or higher than 10000 μg/ml. It can be seen, from these measurement results, that a quantifiable region can be enlarged at least by ten times, compared to the case where the measurement time is not changed according to the concentration range.

### Example 2

In the present example, only the reference point in Example 1 was changed. In Example 1, a sequence with a four-minute measurement time was used as the measurement reference; however, the reference measurement time was set to three minutes in the present example. It was proved that, in the case where Sx1<threshold, a range over which the quantifiable concentration range is enlargeable is enlarged through performing a measurement using a sequence with a long measurement time (10 minutes) suitable for quantitative detection of an antigen concentration in a low concentration region, as denoted by the black circle symbols in FIG. 12.

### Example 3

In the present example, only the reference point in Example 1 was changed. In Example 1, a sequence with a four-minute measurement time was used as the measurement reference; however, the reference measurement time was set to 10 minutes in the present example. It was proved that, in the case where Sx1>threshold, a range over which the quantifiable concentration range is enlargeable is enlarged through performing a measurement using a sequence B with a short measurement time (3 minutes) suitable for quantitative detection of an antigen concentration in a high concentration region, as denoted by the black square symbols in FIG. 12.

### Example 4

In connection with the second embodiment, if Sx1<threshold, the concentration range of the antigen falls in the low-concentration range, and thus a measurement channel (a high-reactivity channel) with a high reactivity suitable for quantification of the low-concentration range, as denoted by the black circle symbols in FIG. 13, was used. As described above, it can be seen, from the measurement of a specimen containing an antigen at a concentration higher than 1000 μg/ml, that the signal intensity of the measurement signal approximates its maximum value. On the other hand, if Sx1>threshold, the concentration range of the antigen falls in the high-concentration range, and thus a measurement channel (a low-reactivity channel) with a low reactivity suitable for quantification of the high-concentration range, as denoted by the white circle symbols in FIG. 13, was used. In this case, it can be seen that the signal intensity of the measurement signal approximates its maximum value at a concentration equal to or higher than 5000 μg/ml. It can be seen, from these measurement results, that a quantifiable region can be enlarged at least by five times, compared to the case where the measurement channel is not changed according to the concentration range.

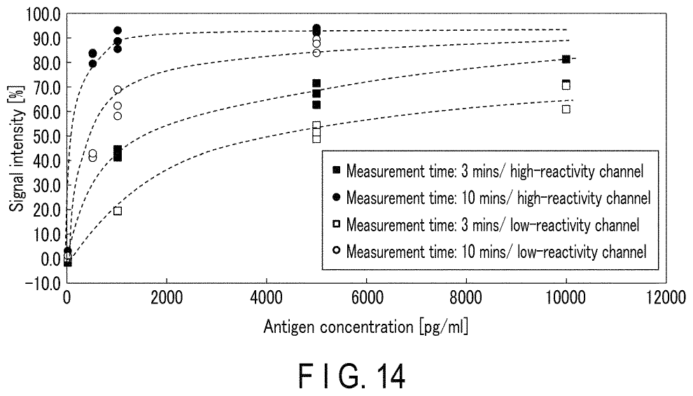

In connection with the third embodiment, if Sx1<second threshold<first threshold, a measurement channel (a high-reactivity channel) with a high reactivity suitable for quantification of the low-concentration range and a measurement sequence with a long measurement time (10 minutes) suitable for quantification of the low-concentration range, as denoted by the black circle symbols in FIG. 14, were used. If second threshold<Sx1<first threshold, a measurement channel (high-reactivity channel) with a low reactivity suitable for quantification of the high-concentration range and a measurement sequence with a long measurement time (10 minutes) suitable for quantification of the low-concentration range, as denoted by the white circle symbols in FIG. 14, were used. If first threshold<third threshold<Sx1, a measurement channel (a low-reactivity channel) with a low reactivity suitable for quantification of the high-concentration range and a measurement sequence with a short measurement time (3 minutes) suitable for quantification of the high-concentration range, as denoted by the white square symbols in FIG. 14, were used. If first threshold<Sx1<third threshold, a measurement channel (a high-reactivity channel) with a high reactivity suitable for quantification of the low-concentration range and a measurement sequence with a short measurement time (3 minutes) suitable for quantification of the high-concentration range, as denoted by the black square symbols in FIG. 14, were used. It can be seen, from these measurement results, that a quantifiable region can be enlarged at least by ten times, compared to the case where a change of the measurement time and selection of the measurement channel are not performed according to the concentration range in the specimen, and that improvement in measurement precision by an increase in a determination index can be expected.

### (Modifications)

The above-described embodiments are described as being applicable to an optical-waveguide immunodetection method that uses magnetic particles. However, the present embodiment is not limited thereto, and may be applicable to various immunoassay methods, no matter whether magnetic particles are employed or not. Examples of the optical measurement technique according to such a modification include immunochromatography and immunonephelometry that does not use magnetic particles. In such an optical measurement technique, a concentration of a reagent added to a specimen, a reagent type, a stirring time, a stirring intensity, a wavelength of irradiation light, and/or a measurement time are included as parameters of the measurement sequence.

According to at least one embodiment described above, it is possible to enlarge the dynamic range related to the concentration and to improve the measurement precision.

The term “processor” used in the above explanation means, for example, circuitry such as a central processing unit (CPU), a graphics processing unit (GPU), an application-specific integrated circuit (ASIC), or a programmable logic device (e.g., a simple programmable logic device (SPLD), a complex programmable logic device (CPLD), or a field programmable gate array (FPGA)). The processor reads programs stored in storage circuitry and executes the programs to implement the corresponding functions. Note that the programs may be directly incorporated in circuitry of the processor, instead of being saved in storage circuitry. In this case, the processor reads the programs incorporated in the circuitry and reads and executes the programs to implement the corresponding functions. On the other hand, if the processor is, for example, an ASIC, the corresponding functions are directly incorporated as logic circuitry in the circuitry of the processor instead of the programs being saved in the storage circuitry. Each processor of the present embodiment is not necessarily configured as a single circuit, and a plurality of independent circuits may be combined into a single processor to realize the respective functions. In addition, a plurality of constituent elements shown in FIGS. 1, 6, and 9 may be integrated into a single processor to implement the corresponding functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Regarding the foregoing embodiments, the appendage of the following is disclosed as one aspect and selective features of the invention.

### (Supplementary Note 1)

1. An immunoassay system, comprising:
a measurement unit configured to measure a measurement target substance contained in a specimen in accordance with a measurement sequence to acquire a measurement signal reflecting a concentration of the measurement target substance; and
processing circuitry configured to calculate an index value related to a fluctuation in intensity of the measurement signal during a first period to select a single measurement sequence to be used in processing during or after the first period in accordance with a concentration range corresponding to the index value of the measurement target substance.

### (Supplementary Note 2)

An immunoassay system, comprising:
a measurement unit configured to measure a measurement target substance contained in a specimen at some or all of a plurality of measurement channels in which an immobilized antibody exhibits different reagent properties to acquire a measurement signal reflecting a concentration of the measurement target substance; and
processing circuitry configured to calculate an index value related to a fluctuation in intensity of the measurement signal during a first period to select, from among the plurality of measurement channels, a single measurement channel to be used in processing during or after the first period in accordance with a concentration range corresponding to the index value of the measurement target substance.

### (Supplementary Note 3)

An immunoassay system, comprising:
a measurement unit configured to measure, in accordance with a measurement sequence, a measurement target substance contained in a specimen at some or all of a plurality of measurement channels in which an immobilized antibody exhibits different reagent properties to acquire a measurement signal reflecting a concentration of the measurement target substance; and
processing circuitry configured to calculate an index value related to a fluctuation in intensity of the measurement signal during a first period to select a single measurement sequence and a single measurement channel to be used in processing during or after the first period in accordance with a concentration range corresponding to the index value of the measurement target substance.

### (Supplementary Note 4)

The immunoassay system according to any one of Supplementary Notes 1 to 3, wherein
the processing circuitry quantifies the measurement target substance based on a measurement signal acquired during a second period which is during or after the first period.

### (Supplementary Note 5)

The immunoassay system according to supplementary note 4, comprising:
a storage device configured to store information on a calibration line according to a type of a substance and/or information on the calibration line, wherein
the processing circuitry is configured to quantify the measurement target substance based on the measurement signal acquired during the second period and the calibration line corresponding to the measurement target substance.

### (Supplementary Note 6)

The immunoassay system according to any one of supplementary notes 1 to 3, wherein
the measurement unit includes:
   a substrate which has translucency and on which a first substance that specifically binds to the measurement target substance is fixed;
   a magnet configured to apply a magnetic field for moving magnetic particles to which a second substance that specifically binds to the measurement target substance is bound; and
   an optical instrument configured to detect light made incident on the substrate, propagating through the substrate, and emitted from the substrate, and to output an output signal of the detected light as the measurement signal.

### (Supplementary Note 7)

The immunoassay system according to supplementary note 6, wherein
the first period is set to a local period including an arrival time of a peak of the intensity of the measurement signal upon start of a lower-field application, a local period including an arrival time of a jump up in the intensity of the measurement signal upon termination of the lower-field application, and/or a local period during which the intensity of the measurement signal decreases upon the termination of the lower-field application.

### (Supplementary Note 8)

The immunoassay system according to supplementary note 6, wherein
the first period is set to: a local period within a period ranging from a start time of the measurement signal by the measurement unit, through a start time of a lower-field application, to a termination time of the lower-field application, the local period including an empirical arrival time of a peak; a period ranging from the termination time of the lower-field application to a time when a jump up can be detected; and/or a period ranging from the time when a jump up can be detected to an estimated convergence time when the decrease in the intensity of the measurement signal is estimated to converge.

### (Supplementary Note 9)

The immunoassay system according to supplementary note 1 or 3, wherein
the processing circuitry is configured to:
   select, upon determining, based on a comparison with a threshold that varies according to the index value and the first period, that the concentration range is a first concentration range, the single measurement sequence configured of one or more measurement parameters suitable for quantification of the first concentration range; and
   select, upon determining that the concentration range is a second concentration range lower than the first concentration range, the single measurement sequence configured of one or more measurement parameters suitable for quantification of the second concentration range.

### (Supplementary Note 10)

The immunoassay system according to supplementary note 1 or 3, wherein
an application time of a field to a complex containing the measurement target substance, an intensity of the field, and/or a spontaneous precipitation time of the complex are used as parameters for the measurement sequence in a case of an optical-waveguide immunodetection method which uses magnetic particles.

### (Supplementary Note 11)

The immunoassay system according to supplementary note 1 or 3, wherein
the measurement sequence is, in a case of an optical measurement technique which does not use magnetic particles, a concentration of a reagent to be added to the specimen, a type of the reagent, a stirring time, a stirring intensity, a wavelength of irradiation light, and/or a measurement time.

### (Supplementary Note 12)

The immunoassay system according to supplementary note 2 or 3, wherein
the processing circuitry is configured to:
   select, upon determining, based on a comparison with a threshold that varies according to the index value and the first period, that the concentration range is a first concentration range, the single measurement channel having a reagent property suitable for quantification of the first concentration range; and
   select, upon determining that the concentration range is a second concentration range lower than the first concentration range, the single measurement channel having a reagent property suitable for quantification of the second concentration range.

### (Supplementary Note 13)

The immunoassay system according to supplementary note 2 or 3, wherein
the reagent property is a rate of reaction and/or an efficiency of reaction between the antibody and the measurement target substance.

### (Supplementary Note 14)

The immunoassay system according to supplementary note 1, wherein
the measurement unit is configured to measure the measurement target substance using the single measurement sequence in the processing during or after the first period.

### (Supplementary Note 15)

The immunoassay system according to supplementary note 2, wherein
the measurement unit is configured to measure the measurement target substance using the single measurement channel in the processing during or after the first period.

### (Supplementary Note 16)

The immunoassay system according to supplementary note 3, wherein
the measurement unit is configured to measure the measurement target substance using the single measurement sequence and the single measurement channel in the processing during or after the first period.

### (Supplementary Note 17)

The immunoassay system according to any one of supplementary notes 1 to 3, wherein
the measurement unit is configured to optically measure the measurement target substance.

What is claimed is:

1. An immunoassay system, comprising:
a measurement unit configured to measure a measurement target substance contained in a specimen in accordance with a measurement sequence to acquire a measurement signal reflecting a concentration of the measurement target substance; and
processing circuitry configured to calculate an index value related to a fluctuation in intensity of the measurement signal during a first period to select a single measurement sequence to be used in processing during or after the first period in accordance with a concentration range corresponding to the index value of the measurement target substance.

2. An immunoassay system, comprising:
a measurement unit configured to measure a measurement target substance contained in a specimen at some or all of a plurality of measurement channels in which an immobilized antibody exhibits different reagent properties to acquire a measurement signal reflecting a concentration of the measurement target substance; and
processing circuitry configured to calculate an index value related to a fluctuation in intensity of the measurement signal during a first period to select, from among the plurality of measurement channels, a single measurement channel to be used in processing during or after the first period in accordance with a concentration range corresponding to the index value of the measurement target substance.

3. An immunoassay system, comprising:
a measurement unit configured to measure, in accordance with a measurement sequence, a measurement target substance contained in a specimen at some or all of a plurality of measurement channels in which an immobilized antibody exhibits different reagent properties to acquire a measurement signal reflecting a concentration of the measurement target substance; and
processing circuitry configured to calculate an index value related to a fluctuation in intensity of the measurement signal during a first period to select a single measurement sequence and a single measurement channel to be used in processing during or after the first period in accordance with a concentration range corresponding to the index value of the measurement target substance.

4. The immunoassay system according to claim 1, wherein
the processing circuitry quantifies the measurement target substance based on a measurement signal acquired during a second period which is during or after the first period.

5. The immunoassay system according to claim 2, wherein
the processing circuitry quantifies the measurement target substance based on a measurement signal acquired during a second period which is during or after the first period.

6. The immunoassay system according to claim 3, wherein
the processing circuitry quantifies the measurement target substance based on a measurement signal acquired during a second period which is during or after the first period.

7. The immunoassay system according to claim 4, comprising:
a storage device configured to store information on a calibration line according to a type of a substance and/or information on the calibration line, wherein
the processing circuitry is configured to quantify the measurement target substance based on the measurement signal acquired during the second period and the calibration line corresponding to the measurement target substance.

8. The immunoassay system according to claim 5, comprising:
a storage device configured to store information on a calibration line according to a type of a substance and/or information on the calibration line, wherein
the processing circuitry is configured to quantify the measurement target substance based on the measurement signal acquired during the second period and the calibration line corresponding to the measurement target substance.

9. The immunoassay system according to claim 6, comprising:
a storage device configured to store information on a calibration line according to a type of a substance and/or information on the calibration line, wherein
the processing circuitry is configured to quantify the measurement target substance based on the measurement signal acquired during the second period and the calibration line corresponding to the measurement target substance.

10. The immunoassay system according to claim 1, wherein
the processing circuitry is configured to:
select, upon determining, based on a comparison with a threshold that varies according to the index value and the first period, that the concentration range is a first concentration range, the single measurement sequence configured of one or more measurement parameters suitable for quantification of the first concentration range; and
select, upon determining that the concentration range is a second concentration range lower than the first concentration range, the single measurement sequence configured of one or more measurement parameters suitable for quantification of the second concentration range.

11. The immunoassay system according to claim 3, wherein
the processing circuitry is configured to:
select, upon determining, based on a comparison with a threshold that varies according to the index value and the first period, that the concentration range is a first concentration range, the single measurement sequence configured of one or more measurement parameters suitable for quantification of the first concentration range; and
select, upon determining that the concentration range is a second concentration range lower than the first concentration range, the single measurement sequence configured of one or more measurement parameters suitable for quantification of the second concentration range.

12. The immunoassay system according to claim 1, wherein
the measurement sequence is, in a case of an optical measurement technique which does not use magnetic particles, a concentration of a reagent to be added to the specimen, a type of the reagent, a stirring time, a stirring intensity, a wavelength of irradiation light, and/or a measurement time.

13. The immunoassay system according to claim 3, wherein
the measurement sequence is, in a case of an optical measurement technique which does not use magnetic particles, a concentration of a reagent to be added to the specimen, a type of the reagent, a stirring time, a stirring intensity, a wavelength of irradiation light, and/or a measurement time.

14. The immunoassay system according to claim 2, wherein
the processing circuitry is configured to:
select, upon determining, based on a comparison with a threshold that varies according to the index value and the first period, that the concentration range is a first concentration range, the single measurement channel having a reagent property suitable for quantification of the first concentration range; and
select, upon determining that the concentration range is a second concentration range lower than the first concentration range, the single measurement channel having a reagent property suitable for quantification of the second concentration range.

15. The immunoassay system according to claim 3, wherein
the processing circuitry is configured to:
select, upon determining, based on a comparison with a threshold that varies according to the index value and the first period, that the concentration range is a first concentration range, the single measurement channel having a reagent property suitable for quantification of the first concentration range; and
select, upon determining that the concentration range is a second concentration range lower than the first concentration range, the single measurement channel having a reagent property suitable for quantification of the second concentration range.

16. The immunoassay system according to claim 2, wherein
the reagent property is a rate of reaction and/or an efficiency of reaction between the antibody and the measurement target substance.

17. The immunoassay system according to claim 3, wherein
the reagent property is a rate of reaction and/or an efficiency of reaction between the antibody and the measurement target substance.

18. The immunoassay system according to claim 1, wherein
the measurement unit is configured to measure the measurement target substance using the single measurement sequence in the processing during or after the first period.

19. The immunoassay system according to claim 2, wherein
the measurement unit is configured to measure the measurement target substance using the single measurement channel in the processing during or after the first period.

20. The immunoassay system according to claim 3, wherein
the measurement unit is configured to measure the measurement target substance using the single measurement sequence and the single measurement channel in the processing during or after the first period.
